(19) United States
(12) Patent Application Publication
(10) Pub. No.: US 2024/0382675 A1
(43) Pub. Date: Nov. 21, 2024

(54) SYSTEM AND METHOD FOR DETECTING A LEAKING OCCLUDER VALVE IN A PERISTALTIC PUMP

(71) Applicant: CareFusion 303, Inc., San Diego, CA (US)

(72) Inventors: Daniel ABAL, San Diego, CA (US); Brendan John BURGESS, Poway, CA (US); Ramkumar SUBRAMANIAN, San Diego, CA (US)

(21) Appl. No.: 18/690,706

(22) PCT Filed: Sep. 10, 2021
(86) PCT No.: PCT/US2021/049958

§ 371 Date: Mar. 8, 2024

(51) Int. Cl. A61M 5/168; G01M 3/26; G01M 3/40

(52) U.S. Cl. CPC A61M 5/16859; G01M 3/26; G01M 3/40; A61M 2005/16868; A61M 2005/16872

(57) ABSTRACT

An apparatus method, and system for detecting a leaking occluder valve is disclosed. At least one measurement instrument is connected to a fluid within a fluid tubing upstream or downstream of a pump element of an infusion device. The pump element is configured to periodically cause a compression of the fluid tubing and to isolate a downstream portion of the fluid tubing from an upstream portion of the fluid tubing when the pump element is operating under a normal operating condition. A response of the fluid is measured when the pump element is caused to compress the fluid tubing and a determination is made, based on the response whether the compression has fluidically isolated the downstream portion from the upstream portion. One or more shims may be inserted into the occluder valve to move the platen away from the pump element during the test to determine a degree of fault.

### BACKGROUND

Large volume pumps (LVPs) may be used to deliver of 100 milliliters or more of a fluid from a single container. LVPs are subject to maintenance for various failures over time. One potential mode of failure includes incomplete occlusion of the intravenous (IV) tubing by the peristaltic pump mechanism. For example, the fingers that pinch the tubing to drive fluid within the tubing may not be fully compressing the tubing or, in some instances, may overcompress the tubing.

To resolve incomplete occlusion of an IV line, current methods include running a flow rate accuracy test. However, these tests typically measure the response of the entire system. As such, any deviations in the flow rate can be the result of one or many factors such as inaccurate motor speed, restrictive fitments, and testing setup issues and the like. Consequently, these tests can lead to extensive troubleshooting to replace parts until the root cause of the problem is identified. This added workload represents time and cost wasted to determine a cause. Currently there are no adequate test systems, devices, or methods to identify leaking valves or fingers quickly and efficiently.

### SUMMARY

There is a need to verify the proper operation of LVP infusion pumps with regards to leakage of the occluder valves in pumps with multiple occlusion valves and fingers, as well as leaking in a peristaltic pump mechanism. Accordingly, the subject technology provides an apparatus and method for efficiently identifying occluder valves that are leaking.

According to various implementations, a method includes fluidly connecting at least one measurement instrument to a fluid within a fluid tubing upstream or downstream of a pump element of an infusion device, the pump element configured to periodically cause a compression of the fluid tubing, the compression fluidically isolating a downstream portion of the fluid tubing from an upstream portion of the fluid tubing when the pump element is operating under a normal operating condition; causing the pump element to cause the compression of the fluid tubing; measuring, with the at least one measurement instrument, a response of the fluid when the pump element is caused to compress the fluid tubing; and determining, based on the measured response, whether the compression has fluidically isolated the downstream portion from the upstream portion when the pump element is caused to compress the fluid tubing.

In some implementations, the at least one measurement instrument comprises a first electrode and a second electrode, and the method further comprises fluidically connecting the first electrode to a fluid within upstream portion of the fluid tubing; fluidically connecting the second electrode to a fluid within the downstream portion of the fluid tubing; applying a voltage across the first and second electrodes; and determining whether the compression has fluidically isolated the downstream portion from the upstream portion based on whether a current is generated by the applied voltage. The method may further comprise detecting the current when the pump element is caused to compress the fluid tubing; and determining that the compression fluidically isolated the downstream portion from the upstream portion based on detecting the current.

In some implementations, the at least one measurement instrument comprises a pressure sensor, and the method further comprises measuring, with the pressure sensor, a pressure of the fluid when the pump element is caused to compress the fluid tubing; and determining whether the compression has fluidically isolated the downstream portion from the upstream portion based on the measured pressure. In some implementations, the compression is determined to fluidically isolate the downstream portion from the upstream portion when the measured pressure satisfies a threshold pressure, and the method further comprises determining that the measured pressure satisfies the threshold pressure; and determining that the compression fluidically isolated the downstream portion from the upstream portion based on determining that the measured pressure satisfies the threshold pressure. The infusion device may include occluder valve, the occluder valve comprising the pump element and being configured to compress the pump element against a platen of the infusion device to compress the fluid tubing. In this regard, the method may further comprise iteratively inserting one or more shims between the platen and a portion of the occluder valve to move the platen away from the pump element; and causing the pump element to cause the compression of the fluid tubing while each respective shim of the one or more shims is inserted between the platen and the portion of the occluder valve, wherein the response of the fluid is measured for each respective shim.

According to various implementations, the method includes determining that the downstream portion is currently isolated from the upstream portion; iteratively inserting the one or more shims, while measuring the response with the at least one measurement instrument, until the downstream portion is no longer isolated from the upstream portion when the pump element is caused to compress the fluid tubing; determining a size of a respective shim, of the one or more shims, that causes the downstream portion to no longer be isolated from the upstream portion; and determining the pump element is faulty when the size of the shim satisfying a predetermined threshold size for the pump element.

According to various implementations, a test infusion set comprises a fluid carrying compressible tubing prefilled with a fluid and fluidically sealed at each end; a pair of measurement devices, each measurement device being integrated at a respective end of the fluid carrying compressible tubing; and at least one measurement instrument integrated with the fluid carrying compressible tubing and in fluid communication with the fluid, wherein the at least one measurement instrument is configured to measure a response of the fluid when the fluid carrying compressible tubing is compressed by a pump element of an infusion device and to communicate the measured response to a measurement device remote from the test infusion set.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of an example infusion device showing an infusion set in place within the infusion device, according to various aspects of the subject technology.

FIG. 2A depicts an example pumping mechanism of an infusion pump including two occluder valves, according to various aspects of the subject technology. FIG. 2B depicts an example delivery pattern over time for the example pumping mechanism of FIG. 2A.

FIG. 3 depicts an example test setup used for detecting a leaking occluder valve, according to various aspects of the subject technology.

FIGS. 4A and 4B depict example electrical circuits, according to various aspects of the subject technology.

FIG. 5 depicts an example test segment for detecting a leaking occluder valve, according to various aspects of the subject technology.

FIG. 6 depicts an example occluder valve, including an occluder element, according to various aspects of the subject technology.

FIG. 7 depicts an example process for detecting a leaking occluder valve, according to aspects of the subject technology.

FIG. 8 is a conceptual diagram illustrating an example electronic system for detecting a leaking occluder valve, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

A broken platen or broken boss (e.g., door releasing boss or pushing boss) may lead to an unregulated flow scenario where the occluder mechanism does not completely pinch the pumping segment and creating a deviation from the set flow rate. The subject technology provides a system and method that can be utilized during pump maintenance. According to some implementations, the subject technology uses an electric current within the IV set to determine whether one or more occluder valves of the pump are faulty. According to various implementations, the subject technology uses a pump's embedded pressure/force sensor signal to identify unregulated flow and to determine whether one or more occluder valves are faulty. In some implementations, the system and method includes use of a stressor fixture and/or a special IV set during the analysis of the pump to identify if the occluder and pumping fingers are operating normally.

According to some implementations, the disclosed stressor fixtures include metal shims with different thicknesses that may be attached to the pump platen datum to stress the occluder operating condition. As will be described further, by applying an optimal stressor and by using the pressure signal (median upstream pressure) the overall health of an occluder valve may be determined. According to some implementations, a special tubing set is pre-filled with a fluid and sealed both ends, and having an integrated inline pressure sensor, may be used during the pump's maintenance or debug mode. By exercising the pump at different flow rates, the health of the pump can be identified and the health score assigned. According to some implementations, the special tubing set includes electrodes attached to the set. One electrode may be positioned on the tubing set so that, when the set is loaded in a pump, the electrode is upstream of the pump closer to the fluid container 12 (e.g., bag) and second electrode is positioned on the patient side of the set, downstream of the pump. The electrodes are then used to measure the continuity of fluid.

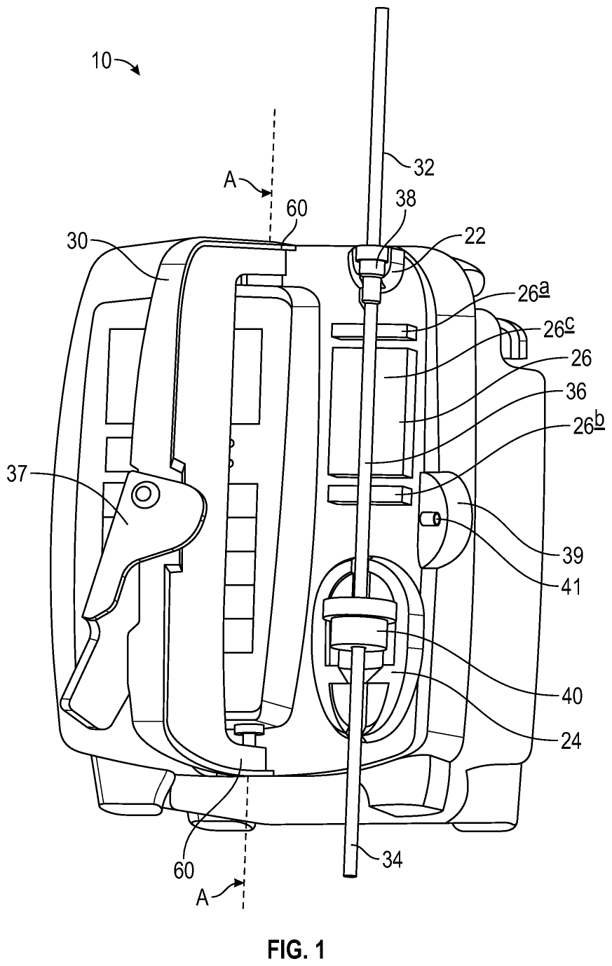

FIG. 1 depicts a perspective view of an example infusion device showing an infusion set in place within the infusion device, according to various aspects of the subject technology. An infusion system for parenteral infusion of a medical fluid to a patient comprises a pump unit, a major part of which comprises a housing which accommodates, in manner known per se, a cam system (not shown) controlling a plurality of fingers of a peristaltic pumping mechanism, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls, all in manner known per se.

As shown in FIG. 1, the infusion device 10 may include a door 30 or face plate which may be opened to reveal the internal loading mechanism for an infusion set. Within the housing of the infusion device (e.g., behind the door or face place), the infusion device includes a pumping segment 36 including a group of serially-aligned pumping elements configured to compress an elongated compressible channel of the infusion set, when loaded within the pumping segment. The pumping segment includes a group of serially-aligned pumping elements (e.g., occluders and/or pumping finger(s)) configured to compress the elongated compressible channel (e.g., an IV tubing segment) loaded within the pumping segment.

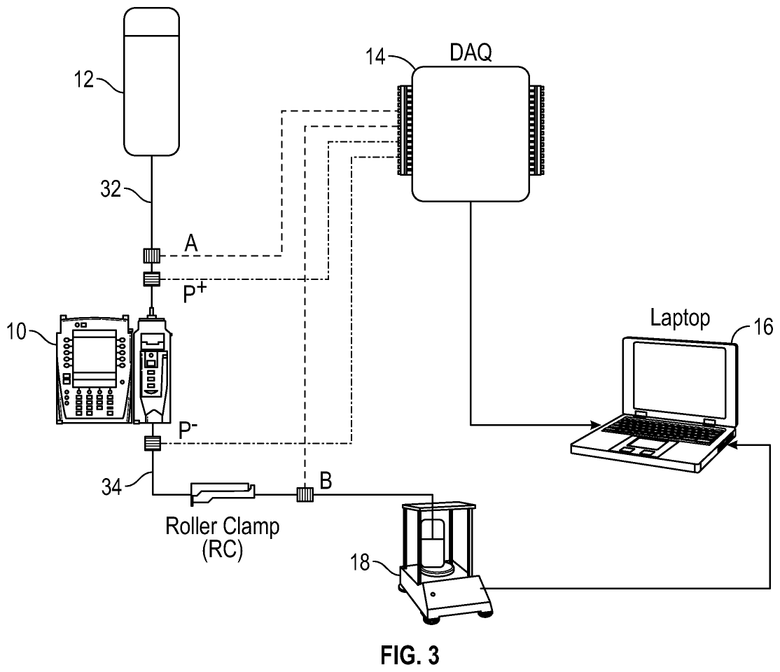

The infusion set includes upper and lower sections 32 and 34 respectively of a tubing, a pumping segment 36 including an intermediate section of resiliently compressible tubing, for example of silicone rubber and, in some implementations, upper and/or lower fittings 38 and/or 40 via which the intermediate tubing section 36 may be connected respectively with a respective upper line 32 and with the lower line 34. In use, each upper line 32 extends upwardly to a source of the medical fluid to be administered whilst the lower line 34 extends from the infusion pump to an infusion needle or the like inserted into the patient. In use, the pumping segment 36 of infusion set is extended across the face or deck of the pump unit so that the fittings 38 and 40 are received in respective brackets 22 and 24 respectively and so that each tubing segment 202, 204 extends over a respective peristaltic assembly 26 as illustrated in FIG. 3. In the depicted example, the infusion set is fitted in place in this fashion whilst the door 30 is in the open position. After the infusion line has been so fitted, the door 30 may be moved to the closed position and is secured by a catch 37 which may include a lever mounted on the outer edge of the door.

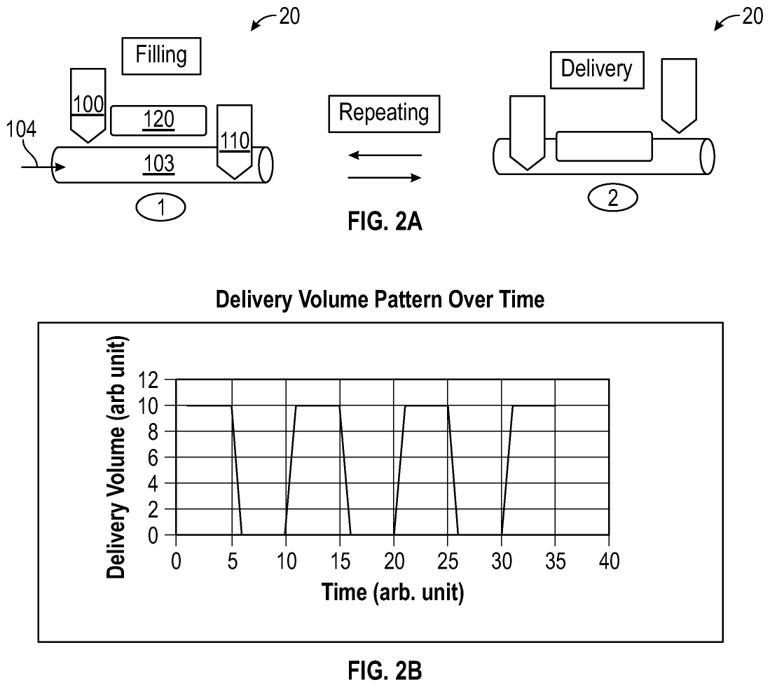

The peristaltic assembly 26 includes respective fingers that are moveable by a cam system (not shown) inwards and outwards from the face or deck 20 of the pump to compress a respective tubing segment against a counter surface or anvil to propel fluid within the infusion line. In order to make it easier to maintain sterile conditions, these fingers may be covered by a thin flexible membrane, (not shown), sealed at its edges with respect to the deck. The fingers of the peristaltic assembly 26 periodically press the flexible resilient tubing against the counter surface which may be configured on an opposite side, for example, on an inner portion of the door 30. In the example pump shown, each peristaltic assembly includes an upper occluder 26 aand a lower occluder 26 b which are of a relatively limited extent in the longitudinal direction of the infusion line, and an intermediate finger or pad 26 c, between the upper and lower occluders and which one or more fingers 26 c is extended or elongated in the longitudinal direction of the infusion line. In operation, assuming the fluid is to be propelled downwards, as viewed in FIGS. 2 and, along the infusion line, the peristaltic assembly performs a repeating cycle in which, with the intermediate pad 26 c spaced from the counter surface, the upper occluder 26 a presses the flexible tube against the counter surface or anvil to close the tube at the location of the upper occluder 26 a, the lower occluder 26 b is then withdrawn from the counter surface to open the tube at the location of the lower occluder 26 b, then the intermediate pad or finger(s) 26 c is moved towards the counter surface to drive the fluid in the tube adjacent the intermediate pad 26 c downward along the tube, then the tube is pinched closed again between the lower occluder 26 b and the counter surface, then the upper occluder 26 a is withdrawn from the counter surface and the intermediate finger(s) 26 c withdrawn from the counter surface to draw fresh fluid into the part of the tube adjacent the intermediate finger(s) 26 c.

FIG. 2A depicts an example pumping mechanism 20 of an infusion device 10 including two occluder valves 100, 110 (or 26a, 26b), according to various aspects of the subject technology. A typical peristaltic medical pump for IV infusion delivery has two occluders, a first occluder 100 located upstream and a second occluder 110 located downstream, with a plunger 120 (or 26c) in between. The occluders and plunger coordinate with each other in programmable, sequential steps, controlled by a cam shaft to have two phases: 1) a filling phase, and 2) a delivery phase. The occluders move fluid in a tubing 103 by sequentially compressing the tubing, thereby causing a flow in a direction 104 according to the particular compression sequence of the occluders.

During the medication infusion process, in the filling phase, the upstream occluder 100 lifts to suck the medication into the tubing segment, which creates a pause, followed by the delivery phase to push the fluid out. These sequences can repeat through multiple cycles. To specify, when the plunger of a single plunger/tubing design is lifted from the tubing segment during the filling phase, there will be a disruption in the continuous infusion process. As a result of using this design, the medication delivery will behave with a pulse patten as shown in FIG. 2B.

FIG. 3 depicts an example test setup used for detecting a leaking occluder valve, according to various aspects of the subject technology. An infusion set is loaded into an infusion device 10, as depicted in FIG. 1. Electrodes are introduced into the upstream section 32 of the infusion set and into the downstream section 34 of the infusion set, respectively. In some implementations, the infusion set may include a Y-site at points A and B (not shown), and each electrode may be introduced into the fluid path by way of a respective Y-site. When a fluid is introduced into the tubing, and the flow path between the upstream section 32 and the downstream section is unimpeded (e.g., the occluders are open), the fluid will operate to close an electrical circuit between the electrodes. In this regard, flow continuity may be verified by taking electrical measurements across locations A and B.

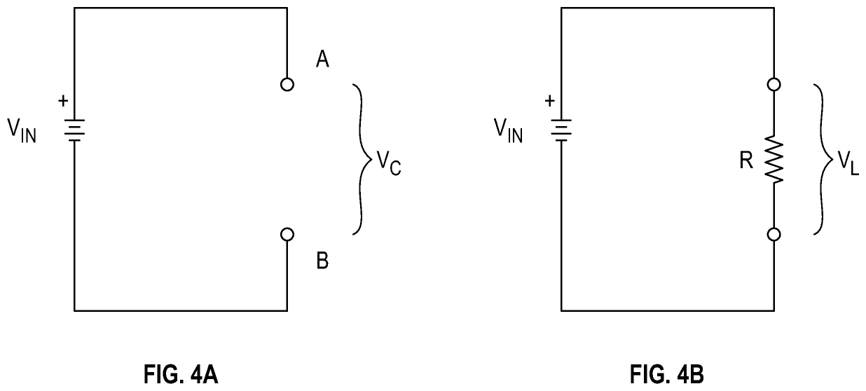

FIG. 4A depicts an electrical circuit representing an open circuit with the voltage Vc being the same as the applied voltage. This represents the occluding valve completely sealing and not providing electrical conductivity. When the electrical pathway between A and B is closed (e.g., electricity is conducting), the system indicates the fluid pathway is open. When the electrical pathway is open between A and B, the system indicates the fluid pathway is closed.

FIG. 4B depicts a second example electrical circuit representing a closed circuit. When the occluder valves are at least partially open, the fluid acts as a conductor, but may include a small resistance R. Thus, a voltage VL measured by the electrodes is determined by:

$$V L = R \cdot I \qquad ( Eq . 1 )$$

Where R and I represent the resistance and current through fluid.

Accordingly, voltage VL (across A and B) is less than the applied voltage as current is flowing through the resistance element which represents the resistivity of the fluid in the IV set. This represents the occluding valve allowing fluid leakage, completing the electrical circuit.

With further reference to FIG. 3, the system may use a power supply (not shown) that provides a low DC voltage such as a nominal 5V DC between locations A and B. The test segment A, B introduces an electrical pathway between the upstream and downstream sides. The set is filled with an electrically conductive fluid. Common Ionic fluids such as a 0.9% saline solution can be used to perform the test. The other components of the IV set such as the fitments and flowstop are made of plastic, which is electrically non-conductive, as well as the pumping segment which is made of silicone, thermoplastic elastomer, or other deformable polymer based tubing.

A voltage measurement device 14, such as an oscilloscope, data acquisition system, or a DVM (digital voltage meter), may be used to measure the voltage across the inlet and outlet ports. When the fluid pathway is closed (e.g., due to a complete closure of an occluder valve) there will be no electrical conduction and the voltage measured (VC) will be the same as the input voltage VIN (see FIG. 4A). When the fluid pathway is open (e.g., due to an incomplete closure of an occluder valve) electricity will be conducted by the fluid and the voltage measured across the two points (A and B) will be less than the input voltage (see FIG. 4B). VL is proportional to the fluid electrical resistance, and the associated current flowing through the fluid.

To perform a test, the IV set is loaded, and the electrodes applied, and the pump is set to infuse at a nominal flow rate. As will be described further, in some implementations, a special IV set may be used that integrates the electrodes within the set. As the peristaltic pump mechanism alternates through its cycles, the electrical response from the set may be identified by the voltage measurement device 14, which may include further display and analysis by a computing device 16. If the results show that the voltage measured has decreased anytime during the pump cycle, then this provides indication of one or more leaking occluding fingers.

If an abnormal reading is detected, then an additional troubleshooting step may be used to determine which of the fingers is leaking. For example, for a three, or more finger pump, the pump motor can be rotated to the point where only the upper occluder is closed and the voltage is verified to indicate whether there is leakage. Likewise, the lower occluder can be tested by rotating the motor to the position where only the lower occluder is closed. In this manner, the pump motor may be jogged to different points to identify which finger is leaking. For example, for a twelve finger pump, the motor would be rotated at intervals of 30° and stopped to detect for leakage.

As an example, when a pump is suspected of over infusion, the pump may be returned to a maintenance area, sometimes with the IV sets used when the over infusion was detected. Traditionally, technicians would implement a rate accuracy test by running the pump at different flow rates, measuring using gravimetrics to see if the problem may be reproduced. However, many times the problem cannot be reproduced.

If the pump is operating normally, there should not be an over infusion because one of the occluders is always closed. In other words, there will be no continuous path from the source of fluid to the patient. Using the disclosed method for detecting electrical conductivity, an open condition should be detected (no conductivity) during normal operation. On the other hand, if the peristaltic pump mechanism is defective, an electrically closed circuit will be detected by the measurement device 14 when the previously described electrode test is performed, and a decrease in pressure (beyond a predetermined pressure threshold) will be detected when the previously described pressure test is performed.

When the path is electrically open (and, e.g., fluid is impeded by occlusion), the system may measure a first voltage VC (e.g., 5V) which, in some implementations, may be equal to the input voltage VIN. When the path is electrically closed (e.g., fluid is flowing unimpeded) there may be no resistance and the system measures VL, which may be close or equal zero. When the pump is in normal conditions, one of the downstream or upstream occluder valve 26 a, 26 b may always be closed, and the system should never see an electrically closed path.

The measurement system 14, 16 may be used to visually depict a waveform corresponding to the voltage across A and. Under optimal circumstances, in some implementations, the voltage will remain constant at VC. When there is an occluder fault, the voltage will dip periodically with the opening (and closing) of the faulty valve.

The foregoing system and method provides confirmation that the occluding fingers are completely isolating the upstream and downstream sides of the pump and hence that they are operating as intended, and not allowing leakage.

The collection of measurements and execution of one or more steps may be controlled by a microcontroller or other processing device specifically configured by machine-executable instructions stored in a data storage device. For example, if the initial voltage values indicate a potential leak, the microcontroller may transmit a control message to the pump to jog the pump motor through a sequence of positions to identify which finger may be leaking. The microcontroller may store a log of the voltages and determination results in association with an identifier for the pump to provide an auditable record of the pump state and/or tests performed.

While the subject technology described herein is described with regard to occluder valves and occluder elements, the subject technology is equally applicable to other systems which provide a compression to a fluid filled tubing. For example, a peristaltic pumping mechanism having a multitude of fingers may be checked using the subject technology in the same way previously described. Also, the cam controlling the fingers may be controlled to rotate to a position such that only one finger is engaged against the tubing. In this manner, each finger may be tested individually according to the rotation of the cam.

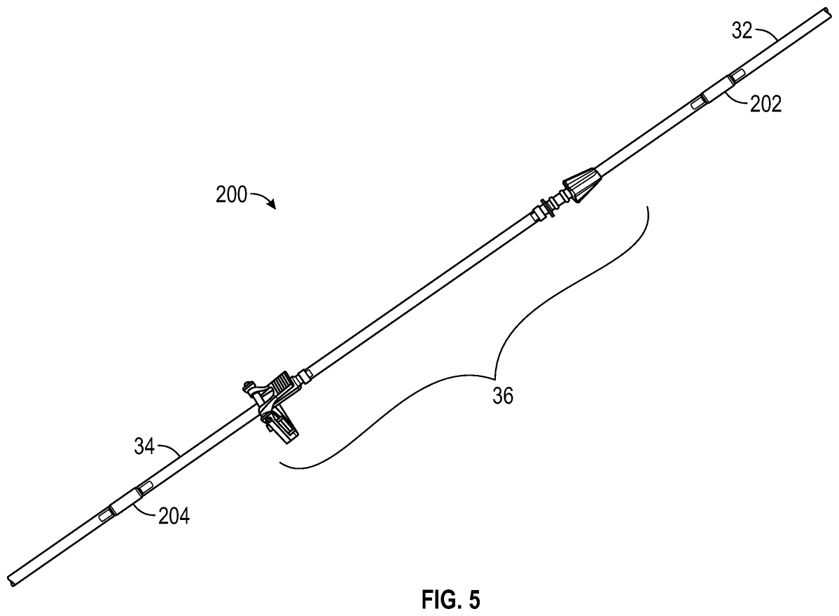

In some implementations, pressure sensors P+, P− may be substituted for the foregoing electrodes. When the test system is configured as depicted in FIG. 3, an upstream static pressure is created by way of the fluid container, while a downstream pressure is created due to a backpressure from the infusion site and movement of the occluder fingers. These pressures may be detected by pressure sensors P+and P−. According to various implementations, pressure sensors P+and P− may be part of, or associated with, the infusion device 10. In some implementations, the pressure sensors may be included in a dummy set, as described below with regard to FIG. 5, and connected to the measurement device 14.

To perform a pressure test, the IV set is loaded, and the pump is set to infuse at a nominal flow rate. As the peristaltic pump mechanism alternates through its cycles, the pressure response from the set may be identified by the measurement device 14, which may include further display and analysis by a computing device 16. The stepping motor creates a signal in the line which is picked up by the upstream and downstream pressure sensors. A pressure wave may be displayed indicating when the pressure is shown to increase or decrease. A pressure decrease beyond a threshold decrease in pressure may be an indication of one or more leaking occluding fingers.

In some implementations, the system may include a fluid measurement device 18 to determine the amount of fluid leaking through the occluder valves. Accordingly, the amount of fluid in fluid measurement device 18 at any particular time, may facilitate a determination of a level of occluder fault (e.g., by the amount of fluid detected).

FIG. 5 depicts an example test segment for detecting a leaking occluder valve, according to various aspects of the subject technology. The test segment 200 includes two fitments 202 and 204 in-line with the fluid path of the segment 200. In the depicted example, each fitment is removably configured to be inserted between two tubing segments 32, 34, 36. Each fitment 202, 204 may include a body providing a fluid path therethrough, with each end of the body including a sealing connector (e.g., male connection point) for connecting fluidically sealing the body to respective tubing segments.

In some implementations, the fitments 202, 204 may be electrodes. In some implementations, the fitments may be electrically conductive fitments on upstream and downstream sides of set. In some implementations, the electrodes 202, 204 may be implemented as removable, electrically conductive fitments. In some implementations, an inner portion of the body may have an electrode embedded therein that, when fluid is passing through the body, comes in electrical contact with the fluid passing through the body. Each fitment 202, 204 may further include an (optionally removable) electric lead for connection to the previously described voltage measurement device 14. In other implementations, electrodes 202, 204 may be integrated with a uniformly constructed test segment 200 (e.g., without removable fitments).

As briefly described with regard to FIG. 3, in some implementations, pressure sensors may be used to supplement or replace the electrodes. In this regard, each measurement fitment 202, 204 may include a pressure sensor. In this regard, as the peristaltic pump mechanism alternates through its cycles, the pressure response P+, P− from the set may be identified via each pressure sensor within fitments 202, 204 by a pressure measurement device 14, which may include further display and analysis by a computing device 16.

In some implementations, the previously described test segment 200 may be a dummy test set, prefilled with a fluid and fluidically sealed (e.g., hermetically sealed) at each end, for example, with each fitment operating to seal the fluid within the segment. In this regard, the segment may be used without the need for a fluid container 12 or drain system 18. In this regard, the segment may be used to form the electrical circuit without the need for a fluid container 12 or drain system 18. Instead of loading a regular IV set, the technician may load the dummy set and close the door 30. Each end of the dummy set may include an electrode, as previously described, or may include an inline pressure sensor P+, P−. Segments 200 may be constructed in different sizes and optimal pressure responses predetermined prior based on testing under optimal conditions. In this manner, when an infusion device 10 is under test, the pressure responses seen by measurement device 14 may be compared to the predetermined pressure responses for the particular set 200 used.

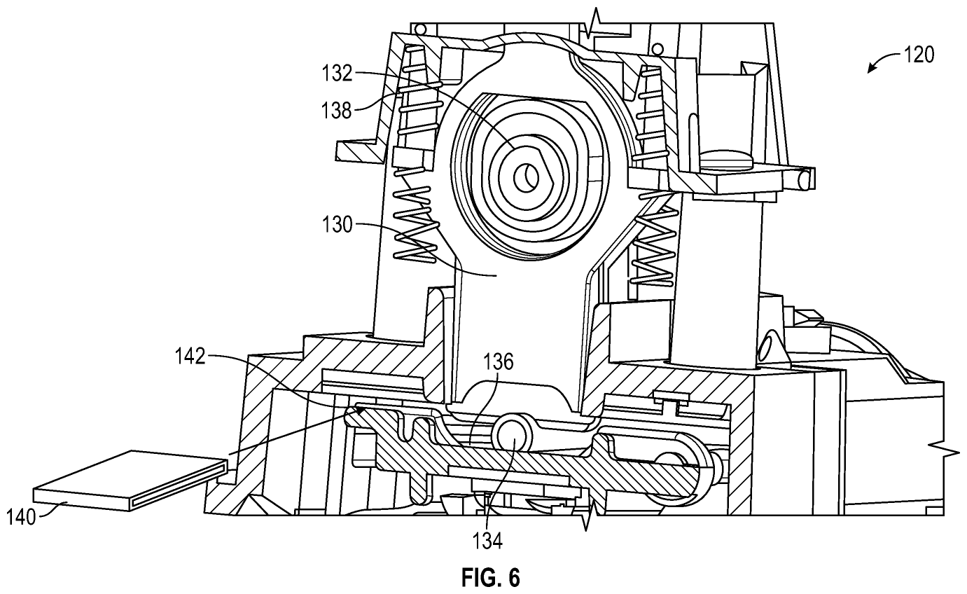

As will be described further with regard to FIG. 6, metal shims (or “stressors”) with different thicknesses may be attached to the pump platen datum to stress the occluder operating condition. Each shim may be attached to the platen to interface with the bottom datum of the occluder and prevent the platen from closing completely. With the shim applied, the electrical conductivity, or the upstream and downstream pressures, may be measured, as described above. In some implementations, a set of shims may be provided with a dummy test set. The size of each shim in the set may be calibrated for the particular set. In some implementations, the set of shims may be connected to the infusion set (e.g., by one or more wires or fasteners) so that the infusion set, and the shims may be provided as an operable system for diagnosing a pump.

FIG. 6 depicts an example occluder valve 120, including an occluder element 130, according to various aspects of the subject technology. According to various implementations, there are two occluder elements 130; one for the upper occluder 26 a, and one for the lower occluder 26 b. Occluder element 130 is configured to move according to a cam motion of a cam 132 to apply a periodic compression to a flexible infusion line 134 when the flexible infusion line is placed between the occluder element 130 and a plate assembly 136, also termed “platen”. In this regard, the cam motion oscillates the occluder element 130 to move a fluid within the flexible infusion line by periodically compressing the flexible infusion line. Compression springs 138 apply a constant force to the occluder element 130, forcing it against the plate assembly 136, while the cam 132 applies a force at predetermined intervals in an opposite direction, moving the lower portion of the occluder element responsible for compressing the infusion line 134 away from the plate assembly. Each cam 132 may be elliptical in shape and may rotate on an axis off center of the ellipse.

FIG. 6 depicts the occluder element 130 in the top dead center position (top position) with the stroke of the cam is fully upward and with the occluder element furthest apart from the plate assembly 136. A normal occluder element 130 compresses a tubing with a predetermined tolerance. That is, the occluder valve has a certain gap tolerance, or threshold distance between the platen 136 and the occluder element 130 when it is fully extended whereby the occluder element 130 will still fully compress the tubing 134. With brief reference to FIG. 1, the platen 136 may be capable of moving up and down with respect to the valve structure, and may be mechanically connected to the door mechanism of door 30. In this regard, the platen 136 may move away from the occluder element when the door 30 is opened, and move to lock in place as depicted in FIG. 6 when the door 30 is closed.

According to various implementations, a shim, also termed a “stressor” 140, is provided for insertion into the platen datum area 142, between the platen 136 and the valve structure. The stressor, based on its thickness, creates an additional artificial gap between the platen and the occluder finger, moving the platen 136 away from the occluder element, generally, and does not allow the occluder to fully press against the platen.

A field technician may employ multiple stressors of different thicknesses to (e.g., iteratively) assess the health of the occluder valve 130. Increasing stressor thickness moves the platen away from the occluder element 130. In this regard, stressors may be inserted between the platen and the platen 136 up to a certain thickness (0.4 mm) without increasing the amount of compression required to fully close the fluid path from the pump to the patient. At some point, as the platen 136 is moved further away, the tubing 136 will no longer be compressed fully by the occluder element 130 and a leakage will occur. In other words, if a stressor at or above a threshold thickness is inserted, the measurements will begin to show a fluid path (e.g., by conducting electricity between the previously described electrodes). In this regard, the threshold on the thickness of the stressor may facilitate a determination of the health of the pump mechanism. A normal occluder will maintain an “open circuit” (no fluid path) up until at least the threshold, while a faulty occluder may create an electrically closed circuit below the threshold.

A pass or failing score may be assigned to the occluder valve based on the point at which the stressor creates a fault condition (e.g., an electrical short or pressure indicating the fault condition). The occluder valve gap tolerance may be known for each type of pump. A pump mechanism in good working order will allow stressors up to the threshold thickness to be inserted before a closed circuit is created. In such situations, the pump may receive a passing score. For example, if the threshold is 0.4 mm and the circuit closes when a 0.4 or 0.5 mm stressor is used, then the occluder valve may receive a passing score. However, once the gap tolerance is exceeded, the occluder valve will no longer compress the tubing enough to cause isolation between the upstream and downstream segments. For example, a stressor having a thickness less than 0.4 mm may cause a fault condition (e.g., using any of the previously described methods). In some implementations, any detection of flow based on a stressor below the threshold thickness will be non-passing.

In implementations where a microcontroller is performing the assessment, an ntifier for the stressor may be provided an input to the assessment. The identifier may be provided using a scannable identifier (e.g., barcode, quick read code, RFID or other wireless tag) of the stressor. Based on the identifier, the microcontroller may identify the thickness of the stressor (e.g., via a look up table associating the identifier with a thickness or other assessment parameters). In some implementations, the stressor may be dynamically adjustable to provide different thicknesses. In such instances, the microcontroller may transmit a control message to the stressor to adjust the thickness according to the test being performed. As discussed above, the microcontroller may store a log of the test configuration such as to audit the status of the pump.

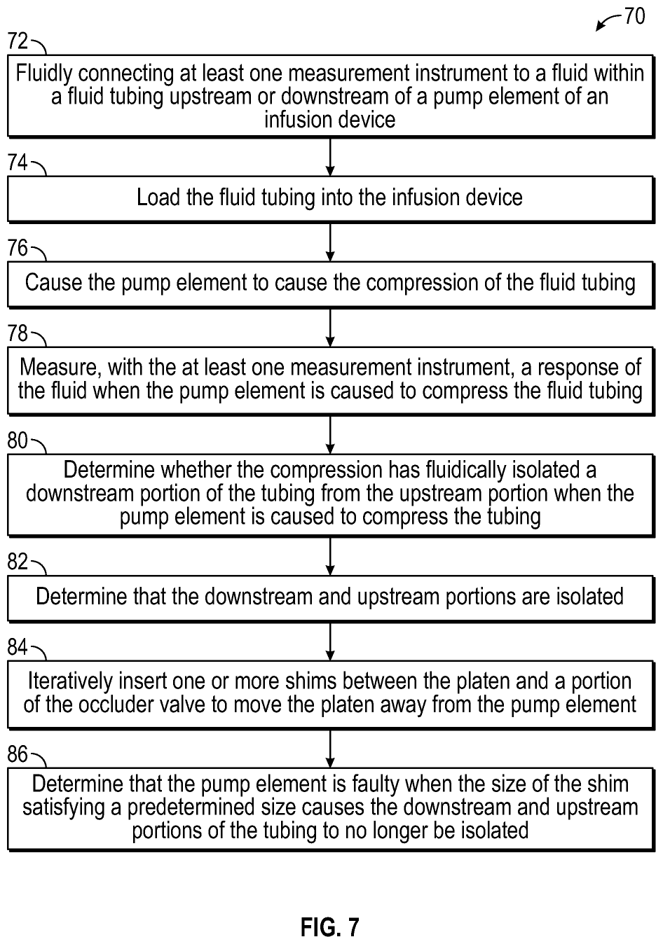

FIG. 7 depicts an example process 70 for detecting a leaking occluder valve, according to aspects of the subject technology. For explanatory purposes, the various blocks of example process 70 are described herein with reference to FIGS. 1 through 6, and the components and/or processes described herein. The one or more of the blocks of process 70 may be implemented, for example, by one or more computing devices including, for example, within infusion device 12. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 70 are described as occurring in serial, or linearly. However, multiple blocks of example process 70 may occur in parallel. In addition, the blocks of example process 70 need not be performed in the order shown and/or one or more of the blocks of example process 70 need not be performed.

In the depicted example, at least one measurement instrument is fluidically connected to a fluid within a fluid tubing upstream or downstream of a pump element of an infusion device (72). For example, the at least one measurement device may be in fluid communication with the fluid. In some implementations, the at least one measurement device is an electrode. In some implementations, a first electrode and a second electrode is used. The first electrode may be fluidically connected to a fluid within upstream portion of the fluid tubing, and the second electrode may be fluidically connected to a fluid within the downstream portion of the fluid tubing. The electrodes may be integrated within the fluid tubing, or may be inserted into each end of the fluid tubing, for example, by way of a y-connector.

In some implementations, the at least one measurement instrument includes a pressure sensor. For example, the fluid tubing may include a respective pressure sensor embedded within the fluid carrying compressible tubing at each end of the fluid carrying tubing. In this regard, each of the pressure sensors may be configured to communicate a pressure reading to a measurement device remote from the test infusion set.

The pump element 130 configured to periodically cause a compression of the fluid tubing, the compression fluidically isolating a downstream portion of the fluid tubing from an upstream portion of the fluid tubing when the pump element is operating under a normal operating condition.

The fluid tubing is loaded into the infusion device (74), and the pump element is caused to compress the fluid tubing (76). For example, the cam of the infusion device may be rotated to a point where the pump element is in a fully closed position at which it is fully compressed (e.g., at a maximum compression) against the tubing.

A response of the fluid is measured, using the at least one measurement instrument, when the pump element is caused to compress the fluid tubing (78).

The measurement device 14 then determines, based on the measured response, whether the compression has fluidically isolated the downstream portion from the upstream portion when the pump element is caused to compress the fluid tubing (80).

When electrodes are implemented, the response may be measured by applying a voltage across the first and second electrodes, and the measurement device 14 may determine whether the compression has fluidically isolated the downstream portion from the upstream portion based on whether a current is generated by the applied voltage. In this regard, the current may be detected when the pump element is caused to compress the fluid tubing.

When a pressure sensor is employed, a pressure of the fluid may be measured when the pump element is caused to compress the fluid tubing; and the measurement device 14 may determine whether the compression has fluidically isolated the downstream portion from the upstream portion based on the measured pressure. In this regard, the compression may be determined to fluidically isolate the downstream portion from the upstream portion when the measured pressure satisfies a threshold pressure. Accordingly, the measurement device 14 determines that the measured pressure satisfies the threshold pressure, and determines whether the compression fluidically isolated the downstream portion from the upstream portion based on determining that the measured pressure satisfies the threshold pressure.

According to various implementations, the infusion device 10 comprises at least a first pump element and a second pump element. To test whether the pump element 130 is operating correctly, the infusion device may be first caused to position the first pump element to be in an open position wherein the fluid flows freely past the first pump element, and to position the second pump element to be in a closed position to cause the compression of the fluid tubing.

The response may then be measured when the second pump element is in the closed position to determine the status of the second pump element. The process may then be reversed to test the status of the first pump element.

In some implementations, the infusion device includes an occluder valve, as shown in FIG. 6. The occluder valve includes the pump element is configured to compress the pump element 130 against a platen 136 of the infusion device to compress the fluid tubing 136. Optionally, one or more shims 140 (also termed “stressors”) may be inserted between the platen 136 and a portion of the occluder valve to move the platen away from the pump element 130, as shown in FIG. 6. In this manner, the pump element 130 is then caused to further cause the compression of the fluid tubing while each respective shim of the one or more shims is inserted between the platen and the portion of the occluder valve. Accordingly, the response of the fluid may be measured for each respective shim.

The process 70 may continue by determining that the downstream portion is currently isolated from the upstream portion (82). This may be accomplished using any of the tests (e.g., electric or pressure) disclosed herein.

The one or more shims are iteratively inserted between the platen 136 and the portion of the occluder valve, while measuring the response with the at least one measurement instrument, until the downstream portion is no longer isolated from the upstream portion when the pump element is caused to compress the fluid tubing (84). A size of the shim that causes the downstream portion to no longer be isolated from the upstream portion is then determined, and the pump element is determined to be faulty when the size of the shim satisfies a predetermined threshold size for the pump element (86).

Many of the above-described devices, systems and methods, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

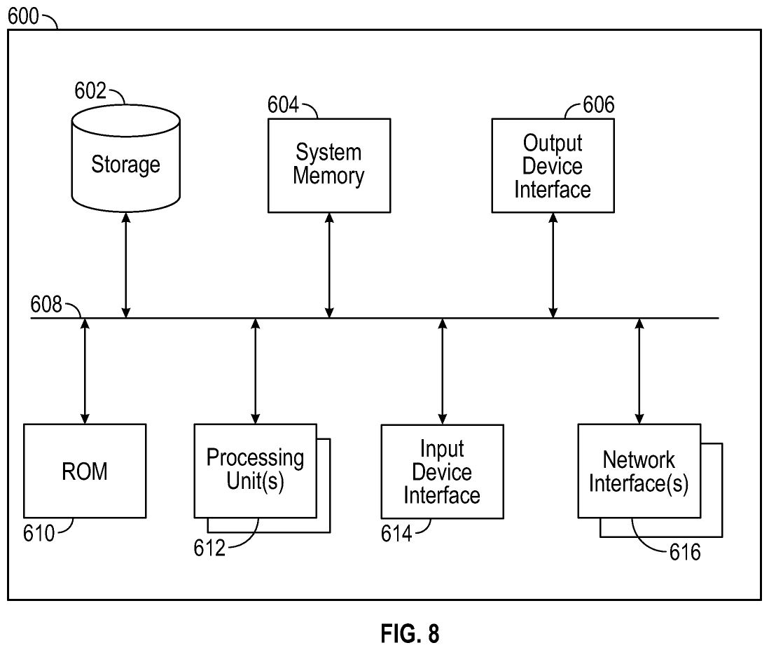

FIG. 8 is a conceptual diagram illustrating an example electronic system 600 for detecting a leaking occluder valve, according to aspects of the subject technology. Electronic system 600 may be a computing device for execution of software associated with one or more components and processes provided by FIGS. 1 to 7, including but not limited to infusion device 10. Electronic system 600 may be representative of a device used in connection or combination with the disclosure regarding FIGS. 1 to 7. In this regard, electronic system 600 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms, web page and server. As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A method, comprising:
maintain a fluid connection between at least one measurement instrument and a fluid within a fluid tubing upstream or downstream of a pump element of an infusion device, the pump element configured to periodically cause a compression of the fluid tubing, the compression fluidically isolating a downstream portion of the fluid tubing from an upstream portion of the fluid tubing when the pump element is operating under a normal operating condition;
receiving the fluid tubing into the infusion device;
causing the pump element to cause the compression of the fluid tubing;
measuring, with the at least one measurement instrument, a response of the fluid when the pump element is caused to compress the fluid tubing; and
determining, based on the measured response, whether the compression has isolated the fluid in the downstream portion from the fluid in the upstream portion when the pump element is caused to compress the fluid tubing.

2. The method of claim 1, wherein the at least one measurement instrument comprises a first electrode and a second electrode, the method further comprising:
fluidically connecting the first electrode to a fluid within upstream portion of the fluid tubing;
fluidically connecting the second electrode to a fluid within the downstream portion of the fluid tubing;
applying a voltage across the first and second electrodes; and
determining whether the compression has isolated the fluid in the downstream portion from the fluid in the upstream portion based on whether a current is generated by the applied voltage.

3. The method of claim 2, further comprising:
detecting the current when the pump element is caused to compress the fluid tubing; and
determining that the compression isolated the fluid in the downstream portion from the fluid in the upstream portion based on detecting the current.

4. The method of claim 2, wherein the infusion device comprises a first pump element and a second pump element, the method further comprising:
causing the infusion device to position the first pump element to be in an open position, wherein the fluid flows freely past the first pump element;
causing the infusion device to position the second pump element to be in a closed position to cause the compression of the fluid tubing; and
measuring, after applying the voltage, the response of the fluid when the second pump element is in the closed position.

5. The method of claim 1, wherein the at least one measurement instrument comprises a pressure sensor, the method further comprising:
measuring, with the pressure sensor, a pressure of the fluid when the pump element is caused to compress the fluid tubing; and
determining whether the compression has isolated the fluid in the downstream portion from the fluid in the upstream portion based on the measured pressure.

6. The method of claim 5, wherein the compression is determined to fluidically isolate the downstream portion from the upstream portion when the measured pressure satisfies a threshold pressure, the method further comprising:
determining that the measured pressure satisfies the threshold pressure; and
determining that the compression isolated the fluid in the downstream portion from the fluid in the upstream portion based on determining that the measured pressure satisfies the threshold pressure.

7. The method of claim 6, wherein the infusion device comprises a first pump element and a second pump element, the method further comprising:
causing the infusion device to position the first pump element to be in an open position wherein the fluid flows freely past the first pump element;
causing the infusion device to position the second pump element to be in a closed position to cause the compression of the fluid tubing; and
measuring the pressure when the second pump element is in the closed position.

8. The method of claim 6, wherein the infusion device comprises the pressure sensor and is operably connected to the at least one measurement instrument, the method further comprising:
communicating, by the infusion device, the measured pressure to the at least one measurement instrument.

9. The method of claim 6, wherein the pressure sensor is integrated into an infusion set and is operably connected to the at least one measurement instrument, the method further comprising:
communicating, by the infusion set, the measured pressure to the at least one measurement instrument.

10. The method of claim 1, wherein the infusion device comprises a occluder valve, the occluder valve comprising the pump element and being configured to compress the pump element against a platen of the infusion device to compress the fluid tubing, the method further comprising:
iteratively receiving one or more shims between the platen and a portion of the occluder valve to move the platen away from the pump element; and
causing the pump element to cause the compression of the fluid tubing while each respective shim of the one or more shims is inserted between the platen and the portion of the occluder valve, wherein a response of the fluid is measured for each respective shim.

11. The method of claim 10, further comprising:
determining that the downstream portion is currently isolated from the upstream portion;
iteratively receiving the one or more shims, while measuring the response with the at least one measurement instrument, until the downstream portion is no longer isolated from the upstream portion when the pump element is caused to compress the fluid tubing;
determining a size of a respective shim, of the one or more shims, that causes the downstream portion to no longer be isolated from the upstream portion; and
determining the pump element is faulty when the size of the shim satisfying a predetermined threshold size for the pump element.

12. The method of claim 1, wherein causing the compression of the fluid tubing comprises causing a compression of a tube segment prefilled with the fluid and sealed at both ends.

13. The method of claim 12, wherein measuring the response of the fluid with the at least one measurement instrument comprises measuring the response using an inline pressure sensor within the fluid tubing at each end of the fluid tubing.

14. The method of claim 12, wherein measuring the response of the fluid with the at least one measurement instrument comprises measuring the response using an electrode at each end of the fluid tubing.

15. The method of claim 1, further comprising:
communicating the measured response to an external measurement device remote from the infusion device and the fluid tubing; and
causing a display of a waveform representative of the measured response on a display device.

16. A test infusion set, comprising:
a fluid carrying compressible tubing prefilled with a fluid and fluidically sealed at each end;
a pair of measurement devices, each measurement device being integrated at a respective end of the fluid carrying compressible tubing; and
at least one measurement instrument integrated with the fluid carrying compressible tubing and in fluid communication with the fluid,
wherein the at least one measurement instrument is configured to measure a response of the fluid when the fluid carrying compressible tubing is compressed by a pump element of an infusion device and to communicate the measured response to a measurement device remote from the test infusion set.

17. The test infusion set of claim 16, wherein the at least one measurement instrument comprises a first electrode and a second electrode, and the response comprises a voltage across the first and second electrodes.

18. The test infusion set of claim 16, wherein the at least one measurement instrument comprises a pressure sensor, and the response comprises a pressure of the fluid within the fluid carrying compressible tubing.

19. The test infusion set of claim 16, wherein the fluid carrying compressible tubing comprises a respective pressure sensor embedded within the fluid carrying compressible tubing at each end of the fluid carrying tubing, each of the pressure sensors configured to communicate a pressure reading to the measurement device remote from the test infusion set.

20. The test infusion set of claim 16, further comprising:
one or more shims coupled to the fluid carrying compressible tubing and each configured to be inserted between a platen and a portion of an occluder valve of the infusion device to move the platen away from a pump element responsible for compressing the fluid carrying compressible tubing when the response is measured by the at least one measurement instrument.
